(19) United States
(12) Patent Application Publication
(10) Pub. No.: US 2025/0111914 A1
(43) Pub. Date: Apr. 3, 2025

(54) PHYSIOLOGICAL MONITOR SENSOR SYSTEMS AND METHODS

(71) Applicant: Masimo Corporation, Irvine, CA (US)

(72) Inventor: Ammar Al-Ali, San Juan Capistrano, CA (US)

(21) Appl. No.: 18/977,618

(22) Filed: Dec. 11, 2024

Related U.S. Application Data
(63) Continuation of application No. 17/662,215, filed on May 5, 2022, now Pat. No. 12,198,790.
Application No. 17/662,215 is a continuation of application No. 16/055,609, filed on Aug. 6, 2018, now abandoned.
Application No. 16/055,609 is a continuation of application No. 13/269,296, filed on Oct. 7, 2011, now abandoned.
(60) Provisional application No. 61/391,067, filed on Oct. 7, 2010.
(60) Provisional application No. 61/393,869, filed on Oct. 15, 2010.

(51) Int. Cl. G16H 10/60; A61B 5/00; A61B 5/0205; A61B 5/1455; G16H 10/40; G16H 50/20; G16H 50/30

(52) U.S. Cl. CPC G16H 10/60; A61B 5/0205; A61B 5/14551; G16H 10/40; G16H 50/30; A61B 5/0015; A61B 5/7264; G16H 50/20

(57) ABSTRACT

A physiological patient monitor can include a front end conditioner that can receive analog sensor signal from a sensor responsive to a person's physiology. The patient monitor can further include an analog-to-digital converter that can convert analog sensor signal to a digital sensor information. The patient monitor can determine one or more patient specific physiological parameters. The patient monitor can further determine a physiological condition of a patient based on processing multiple physiological parameters. The patient monitor can generate a user interface that can included a visual representation of the patient's physiological condition.

### INCORPORATION BY REFERENCE TO ANY PRIORITY APPLICATIONS

The present application is a continuation of U.S. patent application Ser. No. 17/662,215, filed May 5, 2022, titled “PHYSIOLOGICAL MONITOR SENSOR SYSTEMS AND METHODS,” which is a continuation of U.S. patent application Ser. No. 16/055,609, filed Aug. 6, 2018, titled “PHYSIOLOGICAL MONITOR SENSOR SYSTEMS AND METHODS,” which is a continuation of U.S. patent application Ser. No. 13/269,296, filed Oct. 7, 2011, titled “RISK ANALYSIS SYSTEM,” which claims priority benefit under 35 U.S.C. § 119(e) to U.S. Provisional Patent Application No. 61/391,067 filed Oct. 7, 2010, titled “RISK ANALYSIS SYSTEM”and U.S. Provisional Patent Application No. 61/393,869 filed Oct. 15, 2010, titled “DNA RISK ANALYSIS SYSTEM”; all of the above-cited provisional patent applications are hereby incorporated in their entirety by reference herein.

Any and all applications for which a foreign or domestic priority claim is identified in the Application Data Sheet as filed with the present application are hereby incorporated by reference under 37 CFR 1.57.

### BACKGROUND OF THE INVENTION

Pulse oximetry is a widely accepted noninvasive procedure for measuring the oxygen saturation level of arterial blood, an indicator of a person's oxygen supply. A typical pulse oximetry system utilizes an optical sensor attached to a fingertip to measure the relative volume of oxygenated hemoglobin in pulsatile arterial blood flowing within the fingertip. Oxygen saturation (SpO₂), pulse rate and a plethysmograph waveform, which is a visualization of pulsatile blood flow over time, are displayed on a monitor accordingly.

Conventional pulse oximetry assumes that arterial blood is the only pulsatile blood flow in the measurement site. During patient motion, venous blood also moves, which causes errors in conventional pulse oximetry. Advanced pulse oximetry processes the venous blood signal so as to report true arterial oxygen saturation and pulse rate under conditions of patient movement. Advanced pulse oximetry also functions under conditions of low perfusion (small signal amplitude), intense ambient light (artificial or sunlight) and electrosurgical instrument interference, which are scenarios where conventional pulse oximetry tends to fail.

Advanced pulse oximetry is described in at least U.S. Pat. Nos. 6,770,028; 6,658,276; 6,157,850; 6,002,952; 5,769,785 and 5,758,644, which are assigned to Masimo Corporation (“Masimo”) of Irvine, California and are incorporated in their entirety by reference herein. Corresponding low noise optical sensors are disclosed in at least U.S. Pat. Nos. 6,985,764; 6,813,511; 6,792,300; 6,256,523; 6,088,607; 5,782,757 and 5,638,818, which are also assigned to Masimo and are also incorporated in their entirety by reference herein. Advanced pulse oximetry systems including Masimo SET® low noise optical sensors and read through motion pulse oximetry monitors for measuring SpO₂, pulse rate (PR) and perfusion index (PI) are available from Masimo. Optical sensors include any of Masimo LNOP®, LNCS®, SofTouch™ and Blue™ adhesive or reusable sensors. Pulse oximetry monitors include any of Masimo Rad-8®, Rad-5®, Rad®-5v or SatShare® monitors.

Advanced blood parameter measurement systems are described in at least U.S. Pat. 7,647,083, filed Mar. 1, 2006, titled Multiple Wavelength Sensor Equalization; U.S. Pat. No. 7,729,733, filed Mar. 1, 2006, titled Configurable Physiological Measurement System; U.S. Pat. Pub. No. 2006/0211925, filed Mar. 1, 2006, titled Physiological Parameter Confidence Measure and U.S. Pat. Pub. No. 2006/0238358, filed Mar. 1, 2006, titled Noninvasive Multi-Parameter Patient Monitor, all assigned to Cercacor Laboratories, Inc., Irvine, CA (Cercacor) and all incorporated in their entirety by reference herein. Advanced blood parameter measurement systems include Masimo Rainbow® SET, which provides measurements in addition to SpO₂, such as total hemoglobin (SpHb™), oxygen content (SpOC™), methemoglobin (SpMet®), carboxyhemoglobin (SpCO®) and PVI®. Advanced blood parameter sensors include Masimo Rainbow® adhesive, ReSposable™ and reusable sensors. Advanced blood parameter monitors include Masimo Radical-7™, Rad-87™ and Rad-57™ monitors, all available from Masimo. Such advanced pulse oximeters, low noise sensors and advanced blood parameter systems have gained rapid acceptance in a wide variety of medical applications, including surgical wards, intensive care and neonatal units, general wards, home care, physical training, and virtually all types of monitoring scenarios.

### SUMMARY OF THE INVENTION

Multi-parameter patient monitors, while providing physicians with numerical measurements for a variety of specific physiological parameters, do not generally assess a patient's wellness or illness. This shortcoming is especially felt in situations such as a hospital general ward, where the deteriorating health of presumably low-risk patients is often not quickly recognized. Advantageously, a risk analysis system measures a physiological cross-section of parameters and parameter features that are a responsive measure of overall patient wellness or illness.

One aspect of a risk analysis system inputs data regarding a person from sensors and corresponding monitoring equipment, therapeutic devices, laboratory tests and medical records; assesses parameter risks according to physiological parameters derived from the sensor inputs and the impact those parameters have on a person's physiology; and estimates a total risk from a combination of the parameter risks. Total risk is a numerical indication of the likelihood of serious illness or debilitation or, in contrast, the likelihood of wellness or health, the risk analysis system. The risk analysis system has a sensor input responsive to a person's physiology; a physiological parameter derived from the sensor input; and a parameter risk assessment that calculates a corresponding parameter risk from the physiological parameter. A total risk output is derived from the parameter risk.

In various risk analysis system embodiments, the parameter risk assessment comprises sub-parameters defined according to the physiological parameter. Sub-parameter risks are determined from the sub-parameters. A parameter risk calculation determines the parameter risk as a function of the sub-parameter risks. The parameter risk calculation comprise weights assigned to the sub-parameter risks and a weighted average is calculated from the sub-parameter risks and the weights. A sub-parameter risk assessment has a sub-parameter risk function that assigns a sub-parameter risk value to each of the sub-parameters according to measured sub-parameter values and a known relationship between the measured values and underlying physiology represented by the measured values. The sub-parameters comprise a baseline sub-parameter that defines a baseline which tracks proximate a steady state value of the parameter waveform and is relatively unresponsive to transient excursions in the parameter. The physiological parameter is at least one of an oxygen saturation, a pulse rate and a perfusion index.

In various additional risk analysis system embodiments, the sub-parameters comprise an instability sub-parameter responsive to deviations of the parameter from the baseline. The sub-parameters comprise an average slope sub-parameter responsive to absolute values of slopes between adjacent samples of the parameter waveform. Sub-parameter weights correspond to the sub-parameters and the sub-parameters are multiplied by the sub-parameter weights and summed so as to generate a sub-parameter weighted average. The sub-parameters comprise a desat pressure responsive to a relative number and duration of desaturation events. The sub-parameters comprise a pulse rate efficiency responsive to a saturation baseline divided by a pulse rate baseline.

Another aspect to a risk analysis system is inputting sensor signals derived from a person, where the sensor signals are responsive to the person's physiological systems including at least one aspect of the circulatory, respiratory and nervous systems. Physiological parameters are derived from the sensor signals that measure at least some aspect of the functioning of the person's physiological systems. A total risk is calculated from the physiological parameters, which indicates at least a level of urgency in the clinical treatment and care of the person.

In various embodiments the total risk calculation comprises a determination of parameter risks corresponding to the physiological parameters; parameter risk weights corresponding to the sensitivity of the total risk to changes in the underlying parameter; and a sum of the weighted parameter risks. The parameter risk determination defines sub-parameters corresponding to the parameters, determines sub-parameter risks for the sub-parameters and calculates the parameter risks from a weighted sum of the sub-parameter risks. A risk assessment is assigned to each of the sub-parameters. The sub-parameter risks are weighted corresponding to the sensitivity of sub-parameter risks to changes in the underlying sub-parameters.

A further aspect of a risk analysis system comprises partitioning a display into a first panel and a second panel and calculating physiological parameters. Also parameter baselines are calculated and graphs of the physiological parameters and the parameter baselines are presented in the first panel. A total risk is calculated for the physiological parameters and a graph of the total risk is presented in the second panel. In various embodiments, a discrete marker is provided on the display adjacent a parameter label that indicates when the physiological parameter corresponding to the parameter label is calculable within a reasonable probability of error. A risk dot is provided on the display adjacent the parameter label, where the risk dot sized corresponds to the parameter risk. Risk factors responsive to patient specific data are generated that affect the calculation of total risk.

### DESCRIPTION OF THE DRAWINGS

FIG. 1 is a general block diagram of a risk analysis system embodiment;

FIG. 2 is a general block diagram of a total risk calculation;

FIG. 3 is a general block diagram of a physiological parameter contribution to a total risk calculation;

FIG. 4 is a block diagram of an oxygen saturation risk calculation embodiment;

FIGS. 5A-D are graphs illustrating components of an oxygen saturation risk calculation;

FIG. 6 is a block diagram of pulse rate risk calculation embodiment;

FIG. 7 is a block diagram of perfusion index risk calculation embodiment;

FIG. 8 is a block diagram of a total risk calculation embodiment utilizing oxygen saturation, pulse rate and perfusion index risk calculations;

FIG. 9 is a block diagram of a total risk calculation embodiment utilizing oxygen saturation, pulse rate, perfusion index, total hemoglobin, respiration rate, pulse variability index and methemoglobin risk calculations;

FIGS. 10A-B are screenshots of a risk analysis system display embodiment;

FIG. 11 is a screenshot of a risk analysis system display embodiment illustrating risk levels;

FIG. 12 is a screenshot of a risk analysis system display embodiment illustrating both RRa and RRp derived risks;

FIG. 13 is a screenshot of a risk analysis system display embodiment illustrating RRp derived risks; and

FIG. 14 is a detailed block diagram of a risk analysis system embodiment including sensors and a monitor.

### DETAILED DESCRIPTION OF THE PREFERRED EMBODIMENTS

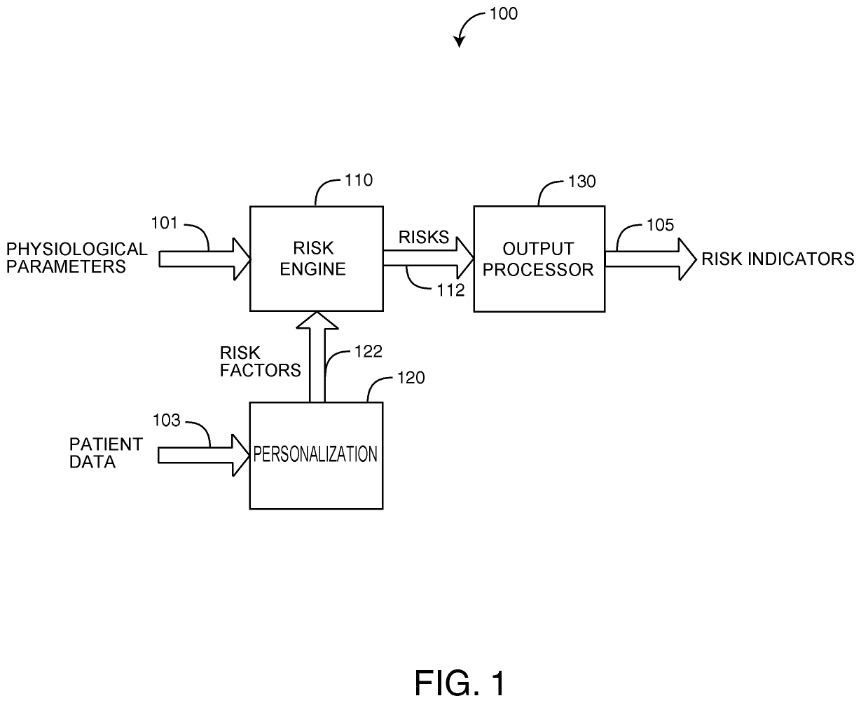

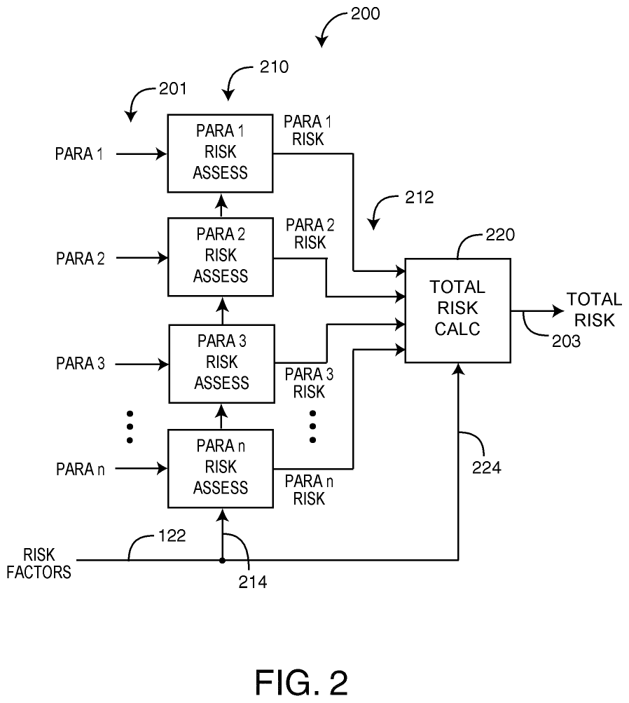

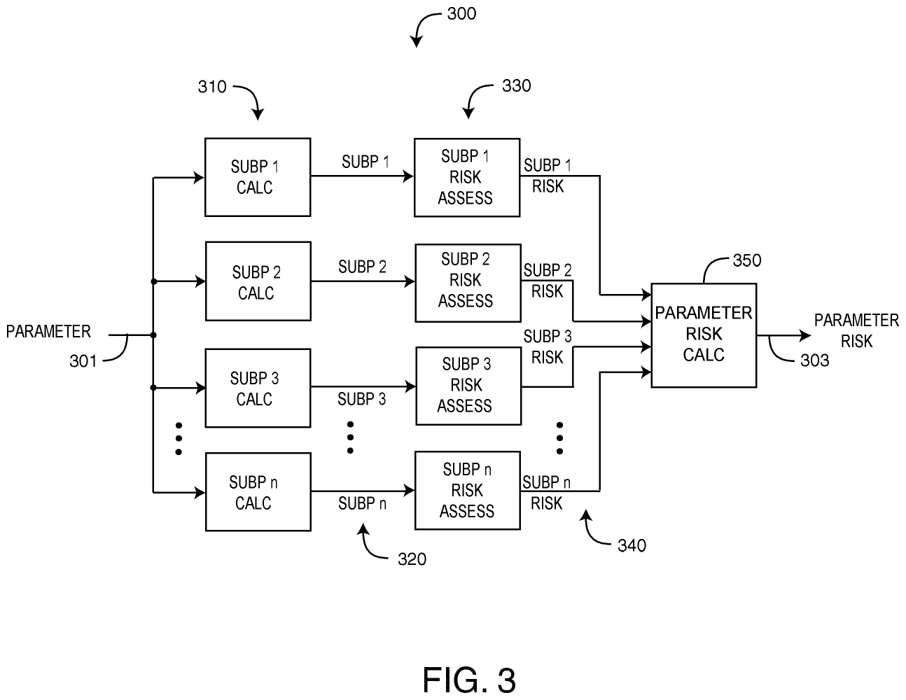

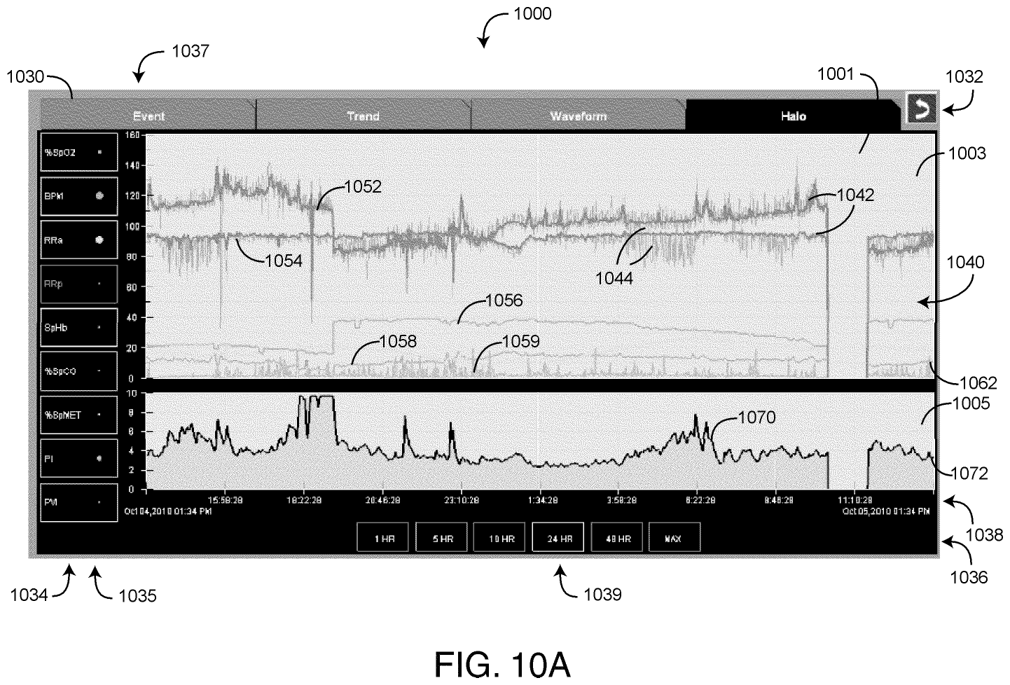

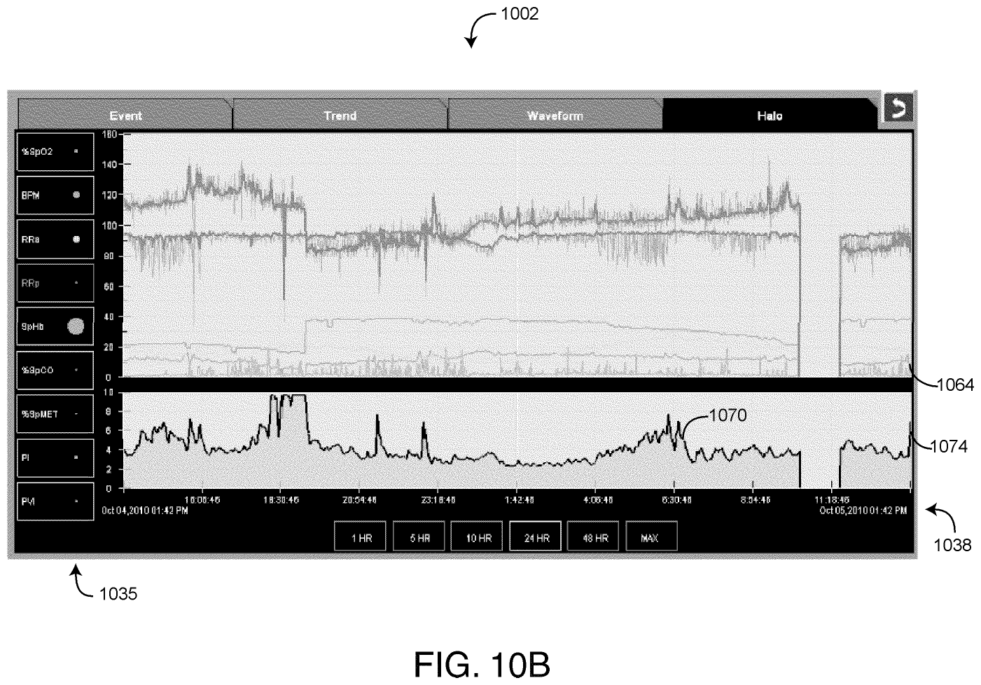

FIG. 1 generally illustrates a risk analysis system 100 having a risk engine 110, an optional personalization 120 and an output processor 130. The risk engine 110 inputs physiological parameters 101 and generates risks 112, as described generally with respect to FIGS. 2-3, below. Personalization 120 inputs patient data 103 and outputs risk factors 122 that modify general calculations of risk according to individual characteristics. Individual characteristics may include a specific past or present illness or medical condition, such as sepsis, cardiac problems or maternity, as examples. Characteristics may also be a general propensity or susceptibility determined by family history or individual DNA, to name a few. The output processor 130 inputs risks 112 and generates one or more risk indicators 105 in various formats including readouts of overall risk or risk component values and/or graphics of risk over time and level of risk, to name a few. Risk indicators 105 are described with respect to FIGS. 10-13, below. Risks 112 and risk indicators 105 can also be transmitted over wired or wireless LAN or WAN to various monitors, devices and displays for use by local or remote medical personnel.

FIG. 2 illustrates a risk analysis embodiment 200 having measured physiological parameter 201 inputs and advantageously generating a total risk 203 output. In an embodiment, total risk 203 is a value from 0 to 1 representing a range of zero percent risk to one hundred percent risk to a person of near-term serious physiological impairment or death due any one or more of disease, injury, surgical complications, drug side-effects or allergic reactions, to name just a few. In various other risk analysis embodiments, risk values may exceed the range 0 to 1 and, accordingly, indicate a relative degree of illness as the risk value increases. In an embodiment, a risk value at or about 1 or greater is an indication to a caregiver of the need for immediate attention to the patient's condition. Similarly, a risk value at or about 1 or greater is an indication that the patient has increasing and immediate vulnerability to permanent physical damage or death due to illness, disease or injury, as examples.

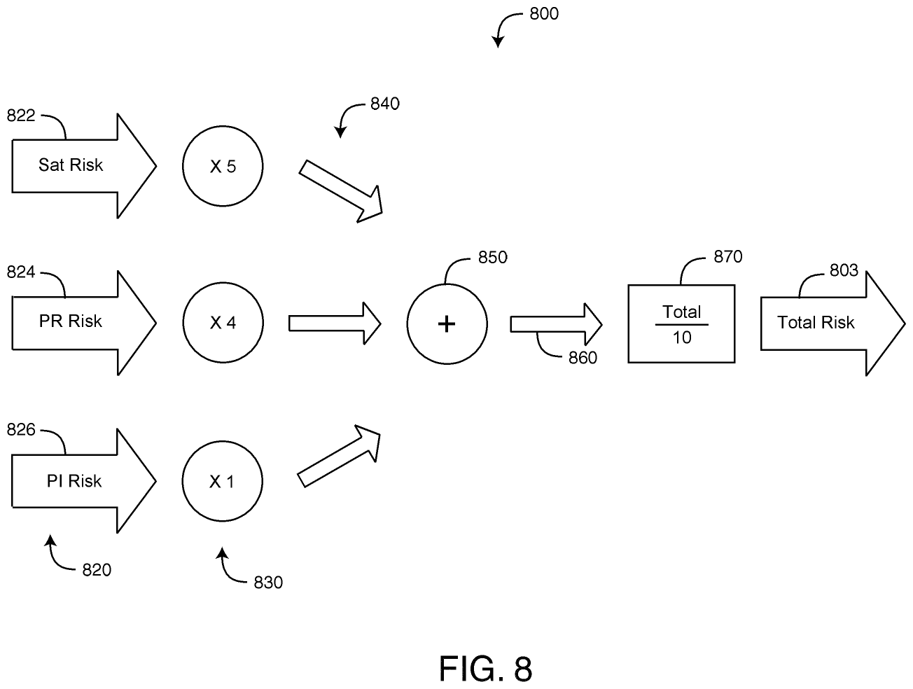

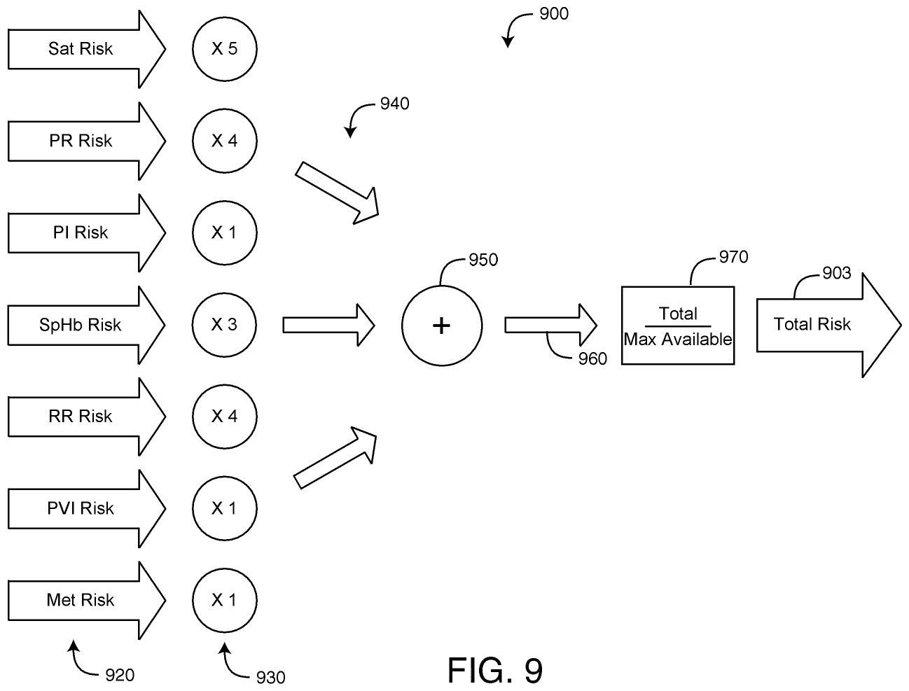

As shown in FIG. 2, a risk assessment 210 is performed for each measured parameter 201 to generate a parameter risk 212. In an embodiment, parameter risk 212 is the contribution to total risk 203 reflected by a measured parameter. In an embodiment, individualized risk factors 122 derived from personalization 120 (FIG. 1) generate one or more parameter risk factors 214 that affect the calculation of one or more of the parameter risk assessments 210 and/or generate a total risk factor 224 that affects the total risk calculation 220. Parameter risk assessment 210 is described in further detail below with respect to FIG. 3. A total risk calculation 220 combines parameter risks 212 to generate a total risk 203. Total risk calculations are described in detail with respect to FIGS. 8-9.

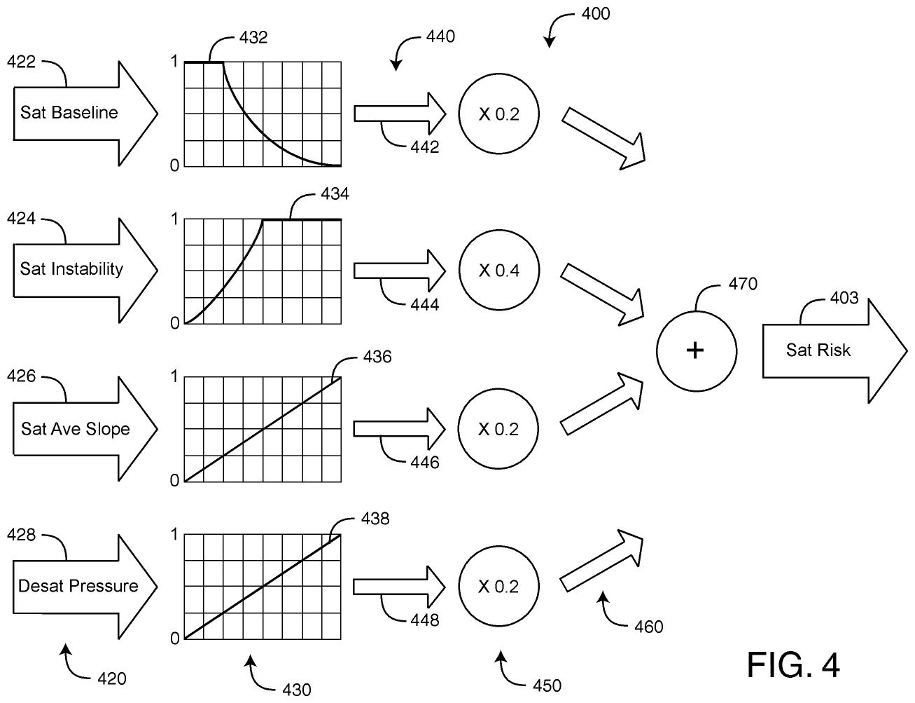

FIG. 3 illustrates a parameter risk assessment 300 embodiment having a parameter input 301, sub-parameter calculations 310, sub-parameter risk assessments 330 and a parameter risk function 350 that generates a parameter risk 303 output. In particular, a set of sub-parameters 320 is advantageously defined for each parameter 301 so as to be inclusive of most, if not all, of the significant physiological phenomena that affect a parameter measurement. This allows parameter risk 303 to be more accurately defined and measured as a set of sub-parameter risks 340. As examples, an instability sub-parameter may be derived from a parameter waveform by measuring the deviations of the parameter waveform from a baseline value. A risk assessment for the instability sub-parameter may be a monotonically increasing function varying from 0 to 1 so as to indicate increasing risk with increasing instability. A parameter risk calculation 350 may be a weighted average of sub-parameter risks 340. In an embodiment, risk factors 122 (FIG. 1) derived from personalization 120 (FIG. 1) affect the calculation of one or more of the parameter risk assessments 210 (FIG. 2) and/or the total risk calculation 220 (FIG. 2). In an embodiment, parameter risk factors 214 (FIG. 2) generate one or more sub-parameter risk factors that affect the calculation of one or more of the sub-parameter risk assessments 330 and/or generate a parameter risk factor that affects the parameter risk calculation 350. Sub-parameters and sub-parameter risk assessment is described in further detail with respect to the examples of FIGS. 4-7, below.

FIG. 4 illustrates a Sat risk calculation 400 embodiment for deriving an oxygen saturation (Sat) risk 403 output from Sat sub-parameter 420 inputs. In particular, an oxygen saturation parameter is factored into oxygen saturation sub-parameters 420 including Sat Baseline 422, Sat Instability 424, Sat Average Slope 426 and Desaturation (Desat) Pressure 428. Sat Baseline 422 and Sat Instability reflect the Sat parameter baseline level and instability, as the labels imply. Sat Average Slope 426 provides a measure of a person's oxygen reserves. If oxygen reserves are low, then oxygen demand results in steep drops in oxygen saturation and, perhaps, corresponding steep rises when oxygen demand is abated. Desat Pressure 428 reflects the relative number and duration of desaturation events.

As shown in FIG. 4, in an embodiment, Sat sub-parameter risk assessment 430 assigns risk values of 0 to 1 (0% to 100% risk) to the sub-parameters. Sat Baseline risk assessment 432 assigns a risk of 1 for very low baseline values. This risk drops rapidly as the baseline value increases from low baseline values, eventually approaching 0 risk at higher baseline values. Sat Instability risk assessment 434 assigns an increasing risk as Sat Instability rises, with a risk of 1 once Sat Instability increases above a certain threshold. Sat Average Slope risk assessment 436 assigns a more or less linear increase in risk as Sat Average Slope rises. Desat Pressure risk assessment 438 assigns a more or less linear increase in risk as Desat Pressure rises.

Further shown in FIG. 4, Sat sub-parameter risks 440 are weighted to yield weighted sub-parameter risks 460. In an embodiment, the sub-parameter risk weights 450 sum to a value of 1. Accordingly, the weighted sub-parameter risks 460 sum to a maximum value of 1. As a result, Sat Risk 403 also varies between 0 to 1 (0% to 100% risk). In an embodiment, Sat Instability 424 is assigned a higher weight 450 than the other Sat sub-parameters 420. In an embodiment, the sub-parameter risk weights 450 are 0.2, 0.4, 0.2 and 0.2 for Sat Baseline risk 442, Sat Instability risk 444, Sat Average Slope risk 446 and Desat Pressure risk 448, respectively. The weighted sub-parameter risks 460 are summed 470 to yield Sat Risk 403. In various other risk analysis embodiments, sub-parameter risk values may exceed the range 0 to 1 and, accordingly, indicate a relative degree of sub-parameter risk as the sub-parameter risk value increases.

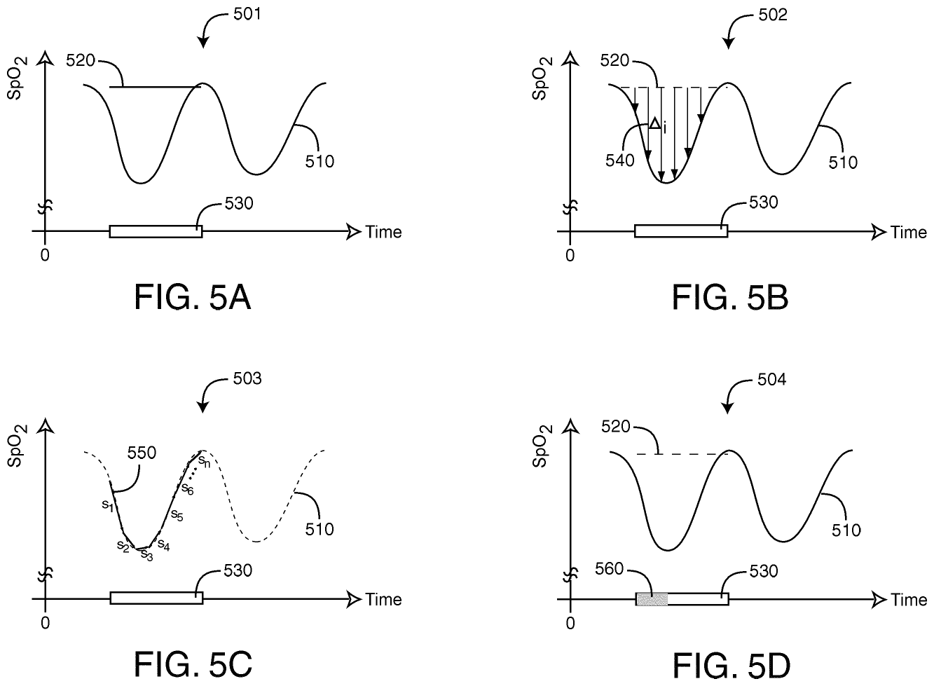

FIGS. 5A-D illustrate an oxygen saturation waveform 510 and features of that waveform that define the Sat sub-parameters accordingly. As shown In FIG. 5A, a Sat baseline 520 is defined to ride near the top of an oxygen saturation waveform 510 so as to be relatively unresponsive to transient dips in oxygen saturation, as shown. Accordingly, a Sat Baseline sub-parameter 422 (FIG. 4) is an average of the baseline values over a time window 530.

As shown in FIG. 5B, a Sat instability is defined according to deviations (Δᵢ) 540 of the instantaneous oxygen saturation waveform 510 from the saturation baseline 520. Accordingly, in an embodiment, a Sat Instability sub-parameter 424 (FIG. 4) is calculated as a weighted sum of the Δᵢ's within the sliding window 530. In an embodiment, the Δᵢ's are exponentially weighted so that smaller Δᵢ's contribute less to the calculation than larger Δᵢ's. In an embodiment, Δᵢ's are limited to a maximum value, such as 10.

As shown in FIG. 5C, a Sat average slope is defined according to absolute values of sᵢ's 550, i.e. slopes between adjacent samples of the oxygen saturation waveform 510. Accordingly, in an embodiment, a Sat Average Slope sub-parameter 426 (FIG. 4) is defined as the average over the sliding window 530 of the absolute value slopes. As shown in FIG. 5D, a desat pressure and corresponding desat pressure sub-parameter are defined according to the percent of time 560, within the sliding window 530, that oxygen saturation is below the baseline and dropping.

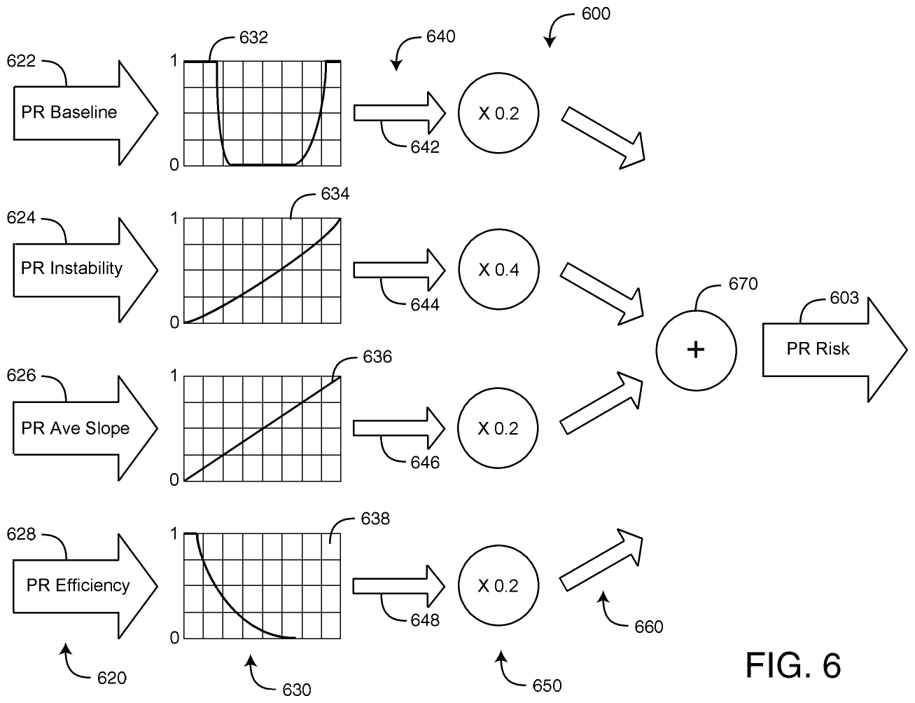

FIG. 6 illustrates a pulse rate risk calculation 600 embodiment for deriving a pulse rate (PR) risk 603 output from PR sub-parameter 620 inputs. A pulse rate measurement is reduced to PR sub-parameter measurements 620 including PR Baseline 622, PR Instability 624, PR Average Slope 626 and PR efficiency 628. Similar to Sat Baseline described above, PR Baseline 622 is defined to ride near the top of pulse rate versus time waveform so as to be relatively unresponsive to transient dips in pulse rate. Accordingly, PR Baseline 622 measurements are an average of the PR baseline values over a sliding time window. PR Baseline risk assessment 632 assigns a risk of 1 for very low or very high PR baseline values and a risk of 0 for PR baseline values within a “sweet-spot” range of pulse rate values considered as normal. PR risk rises rapidly to a risk of 1 as the PR baseline value increases or decreases outside of the PR sweet-spot.

As shown in FIG. 6 and similar to Sat Instability described above, PR Instability 624 is a measure of the deviations of the PR waveform from the PR baseline. In an embodiment, PR Instability 624 is calculated as a weighted sum of the Δ's from the PR baseline within a sliding time window. In an embodiment, these Δ's are exponentially weighted so that smaller Δ's contribute less to the PR Instability risk calculation than larger Δ's and limited to a maximum value so that outliers do not distort the calculation. In an embodiment, PR Instability risk assessment 634 assigns an approximately linearly increasing risk as PR Instability rises.

Also shown in FIG. 6 and similar to Sat Ave Slope described above, PR Ave Slope 626 is a measure of the average over a sliding time window of the absolute values of the slopes sᵢ's defined between adjacent samples of the PR waveform. In an embodiment, PR Average Slope risk assessment 636 assigns a more or less linear increase in risk as PR Average Slope rises.

Further shown in FIG. 6, PR Efficiency 628 is a proxy to cardiac output (dl/min), which is heart stroke (beats per minute)×blood volume (dl per beat). That is, PR Efficiency 628 measures the effectiveness of each pulse in oxygenating the blood. If Sat is high and PR is low, then each pulse is moving a high amount of oxygen. An example is an athlete at rest. In a sick person, the opposite is true. Accordingly, in an embodiment: PR Efficiency=Sat Baseline/PR Baseline. Utilizing these baseline sub-parameters advantageously provides steady-state values as opposed to instantaneous behavior. In an embodiment, PR Efficiency 628 is scaled so as to range between 0-to-1 or equivalently from 0% to 100%. In an embodiment, PR efficiency risk assessment 638 assigns an approximately inversely proportional risk so that PR Efficiency risk 648 drops rapidly as PR Efficiency 628 increases from low efficiency values, eventually approaching 0 risk at higher efficiency values.

Additionally shown in FIG. 6, in an embodiment the PR sub-parameter risk assessment 630 assigns risk values ranging from 0 to 1 (0% risk to 100% risk). PR sub-parameter risks 640 are weighted 650 to yield weighted sub-parameter risks 660. In an embodiment, the sub-parameter risk weights 650 sum to a value of 1. Accordingly, the weighted sub-parameter risks 660 sum to a value of 1. Accordingly, PR Risk 603 also varies between 0 to 1 or 0% to 100% risk. In an embodiment, PR Instability 624 is assigned a higher weight 650 than the other PR sub-parameters 620. In an embodiment, the sub-parameter risk weights 650 are 0.2, 0.4, 0.2 and 0.2 for PR Baseline risk 642, PR Instability risk 644, PR Average Slope risk 646 and PR Efficiency risk 648, respectively. The weighted sub-parameter risks 660 are summed 670 to yield PR Risk 603.

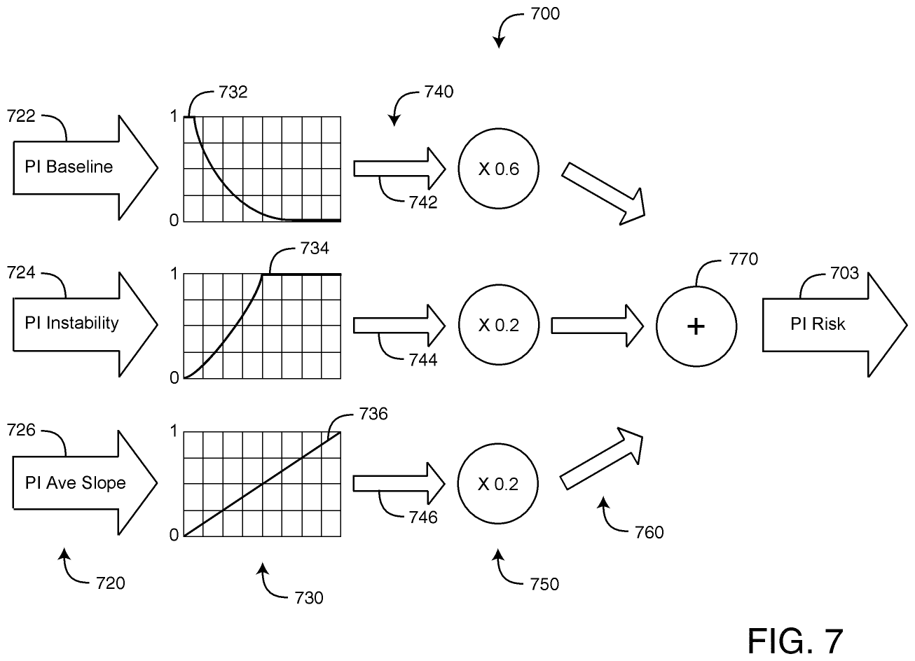

FIG. 7 illustrates a perfusion index risk calculation 700 embodiment for deriving a perfusion index (PI) risk 703 output from PI sub-parameter 720 inputs. A perfusion index measurement is reduced to PI sub-parameter measurements including PI Baseline 722, PI Instability 724 and PI Average Slope 726. Similar to Sat Baseline described above, PI Baseline 722 is defined to ride near the top of a perfusion index versus time waveform so as to be relatively unresponsive to transient dips in perfusion index. Accordingly, PI Baseline 722 measurements are an average of the PI baseline values over a sliding time window. PI Baseline risk assessment 732 assigns a risk of 1 for very low PI baseline values. In an embodiment, PI Baseline risk is roughly inversely proportional to PI Baseline values so that PI Baseline risk drops rapidly as the baseline value increases from low baseline values, eventually approaching 0 risk at higher baseline values.

As shown in FIG. 7 and similar to Sat instability described above, PI Instability 724 is a measure of the deviations of the PI waveform from the PI baseline. In an embodiment, PI Instability 724 is calculated as a weighted sum of the Δ's from the PI baseline within a sliding time window. In an embodiment, these Δ's are exponentially weighted so that smaller Δ's contribute less to the PI Instability risk calculation than larger Δ's and are limited to a maximum value so that outliers do not distort the calculation. In an embodiment, PI Instability risk assessment 734 assigns an approximately linearly increasing risk as PI Instability rises.

Also shown in FIG. 7 and similar to Sat Ave Slope described above, PI Ave Slope 726 is a measure of the average over a sliding time window of the absolute values of the slopes sᵢ's defined between adjacent samples of the PI waveform. In an embodiment, PI Average Slope risk assessment 736 assigns a more or less linear increase in risk as PI Average Slope rises.

Further shown in FIG. 7, in an embodiment the PI sub-parameter risk assessment assigns risk values of 0 to 1 or 0% risk to 100% risk. PI sub-parameter risks 740 are weighted to yield weighted sub-parameter risks 760. In an embodiment, the sub-parameter risk weights 750 sum to a value of 1. Accordingly, the weighted sub-parameter risks 760 sum to a value of 1. Accordingly, PI Risk 703 also varies between 0 to 1 or 0% to 100% risk. In an embodiment, PI Instability 724 is assigned a higher weight 750 than the other PR sub-parameters 720. In an embodiment, the sub-parameter risk weights 750 are 0.6, 0.2 and 0.2 for PI Baseline risk 742, PI Instability risk 744 and PI Average Slope risk 746, respectively. The weighted sub-parameter risks 760 are summed 770 to yield PI Risk 703.

As described with respect to FIGS. 4-7, above, some sub-parameter risks are defined with respect to a deviation from a baseline value, where the baseline value is set at or near the top end of a parameter range. In various other embodiments, sub-parameter risks are defined with respect to a deviation from a baseline value, where the baseline is set, alternatively, at or near the low end of a parameter range or at or near a middle range value of the parameter. Also, in some embodiments, sub-parameter risks may vary over a range of 0 to 1 so as to represent a 0% to 100% sub-parameter risk. In various other embodiments, sub-parameter risks may vary over a range from 0 to a number exceeding 1 so as to indicate a relative degree of sub-parameter risk as sub-parameter risk values increase.

FIG. 8 illustrates a total risk calculation 800 embodiment having Sat Risk 822, PR Risk 824 and PI Risk 826 inputs and generating a Total Risk 803 output. The parameter risks 820 are weighted 830 so that the weighted sum 860 ranges from 0 to 10. Hence, the weighted sum 860 is scaled 870 by 10 to yield a total risk 803 ranging from 0 to 1. In an embodiment, Sat Risk 822 is assigned the highest weight, PR Risk 824 is assigned the next highest weight and PI Risk 826 is assigned the lowest weight so as to reflect the relative contributions of parameter risks to the total risk according to the relative impact on overall health and wellness of a physiological weakness reflected by each of these parameters and their corresponding sub-parameters, as described above. For example, a high Sat risk 822 may correspond to one or more of oxygen saturation instability, low baseline, steep drops/rises in saturation and a relatively high percentage of time below the baseline, any of which would indicate substantial physiological distress. Whereas poor or instable perfusion would be less indicative of severe physiological distress.

FIG. 9 illustrates a total risk calculation 900 embodiment that inputs parameter risks 920 calculated for oxygen saturation, pulse rate, perfusion index, total hemoglobin, respiration rate, pulse variability index and methemoglobin and generates a total risk 903 output. The parameter risks 920 are weighted 930 and the weighted risks 940 are summed 950 to yield a weighted sum 960 ranging from 0 to 19 due to the assigned weights 930. Hence, the weighted sum 960 is scaled 970 by 19 to yield a total risk 903 ranging from 0 to 1. In various other embodiments, total risk 903 can range from 0 to a number exceeding 1. In yet other embodiments, total risk 903 can be presented as a predefined scale ranging over an arbitrary range that monotonically increases or monotonically decreases so as to represent increasing risk, increasing wellness or an increasing measure of clinical significance, to name a few.

FIGS. 10A-B illustrate a risk analysis display 1000 embodiment. FIG. 10B shows parameter and risk displays over a time span 1038 that is eight minutes later than that of FIG. 10A. As shown in FIG. 10A, the display 1000 has a chart area 1001 and a surrounding frame 1030. The frame 1030 has an upper bar 1032 presenting selectable tabs labeled according to the information to display including Event, Trend, Waveform and Halo tabs. The illustrated display shows the Halo tab selected for the display of risk analysis graphs. The side bar 1034 presents parameter value and risk value axes 1037 for the panels 1003, 1005. A lower bar 1036 displays a time axis 1038 for the panels 1003, 1005 and the corresponding time span 1039, which is selectable from 1, 5, 10, 24, 48 and “max” hours. Shown is a 24 hour time span.

In an embodiment, the chart area 1001 is split into an upper panel 1003 and a lower panel 1005. The upper panel 1003 presents parameter graphs 1040 for the parameters listed on the side bar 1034. In an embodiment, the parameters are oxygen saturation (SpO₂), heart rate (BPM), respiration rate-acoustically derived (RRa), respiration rate-pleth derived (RRp), total hemoglobin (SpHb), carboxyhemoglobin (SpCO), methemoglobin (SpMET), perfusion index (PI) and pleth variability index (PVI). The parameter graphs 1040 display parameter values 1044 versus time 1038. Superimposed on the parameter values 1044 are parameter baselines 1042. Parameters and corresponding baselines are described in U.S. patent application Ser. No. 13/037,184 titled Adaptive Alarm System, filed Feb. 28, 2011; a plethysmograph derived respiration rate is described in U.S. patent application Ser. No. 13/076,423 titled Plethysmographic Respiration Processor, filed Mar. 30, 2011; and a physiological monitoring system combining optical and acoustic sensor inputs is described in U.S. patent application Ser. No. 13/152,259 titled Opticoustic Sensor filed Jun. 2, 2011; all patent applications cited immediately above are assigned to Masimo and all are hereby incorporated in their entirety by reference herein.

As shown in FIGS. 10A-B, the lower panel 1005 presents a total risk graph 1070 over all of the parameters listed on the side bar 1034, where total risk is calculated as described above with respect to risks associated with the individual parameters (parameter risk). A side bar 1034 also presents labeled parameters and corresponding parameter risks displayed as a “risk dot” 1035 graphic. In an embodiment, a risk dot 1035 grows larger as the corresponding parameter risk increases and smaller as the parameter risk decreases. This can be seen by comparing FIGS. 10A-B with respect to the SpHb risk dot 1035, which reflects a larger SpHb risk contribution in FIG. 10B as compared with FIG. 10A as the result of a dip in SpHb 1062 (FIG. 10B) as compared with no dip in SpHb 1064 (FIG. 10A). The larger SpHb risk is also reflected in a spike 1074 (FIG. 10B) in total risk 1070. In an embodiment, a tab 1032 can be selected for display of a single parameter in the upper panel 1003 and the associated parameter risk in the lower panel 1005.

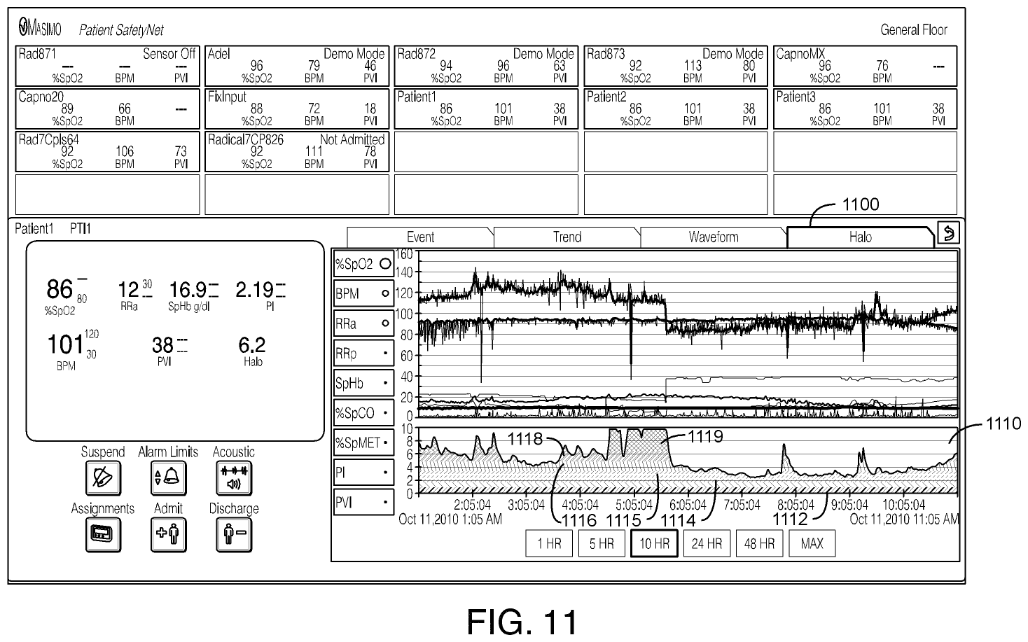

FIG. 11 illustrates a risk analysis display 1100 embodiment having a total risk graph 1110 that displays one or more visual indicators of risk levels, such as stacked colors representing low risk (e.g. green) 1112, medium-low risk 1114 medium risk (e.g. yellow) 1115, medium-high risk 1116, high risk (e.g. orange) 1118 and very high risk (e.g. red) 1119.

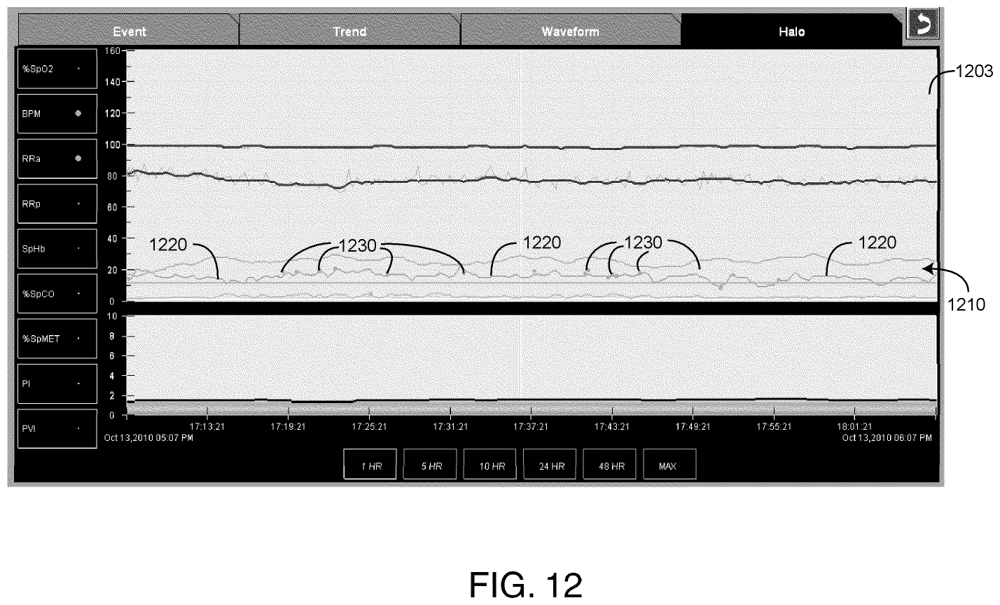

FIG. 12 illustrates a risk analysis display 1200 embodiment having an upper panel 1203 illustrating parameters including respiration rate 1210. Advantageously, RRa (acoustic-derived respiration rate) 1220 and RRp (pleth-derived respiration rate) 1230 are displayed so as to distinguish the manner in which respiration rate is calculated. In particular, RRa 1220 is displayed as a continuous line and RRp 1230 is displayed with discrete markers, such as dots as shown.

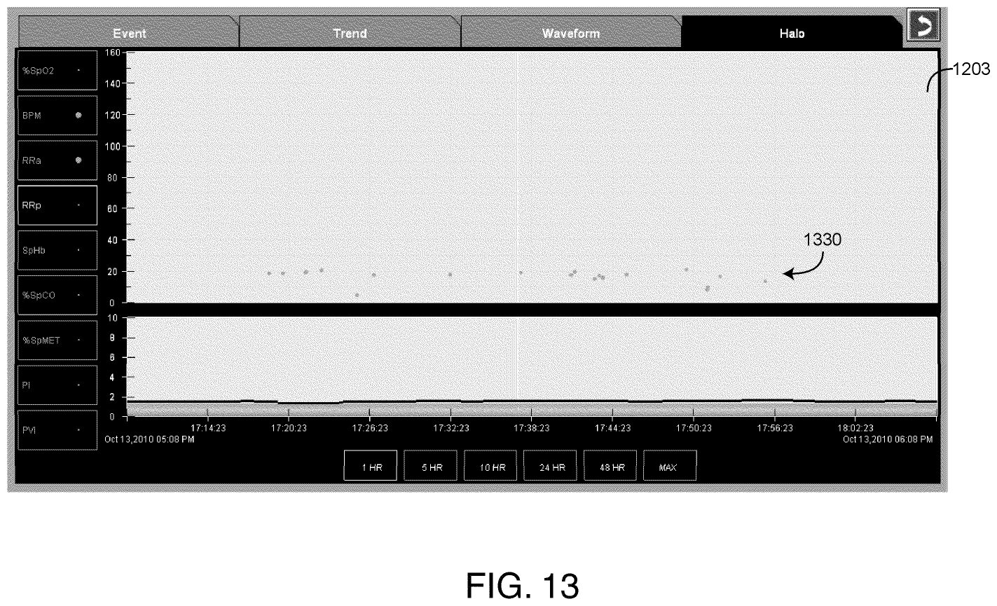

FIG. 13 illustrates a risk analysis display 1300 embodiment having an upper panel 1203 illustrating parameters including RRp (pleth-derived respiration rate) 1330. The discrete markers of RRp, for example dots as shown, advantageously illustrate when the RRp calculation is valid. At other times, i.e. where a marker is missing, the effects of respiration may not be detectable on the pleth or a particular RRp calculation method, or combination of methods, may have more than a reasonable probability of error. Other discrete markers may be X's or symbols, to name a few.

In other embodiments, a risk analysis display may have discrete markers that indicate when a particular test or other measurement is made and, correspondingly, a later time when results of that test or measurement are available. In this manner, a risk analysis system may relate test or measurement results back to the time the test or measurement was taken, adjust a risk calculation accordingly and indicate as much to a caregiver, as described in U.S. Provisional Patent Application No. 61/442,264, filed Feb. 13, 2011, titled Complex System Characterizer, which is hereby incorporated in its entirety by reference herein.

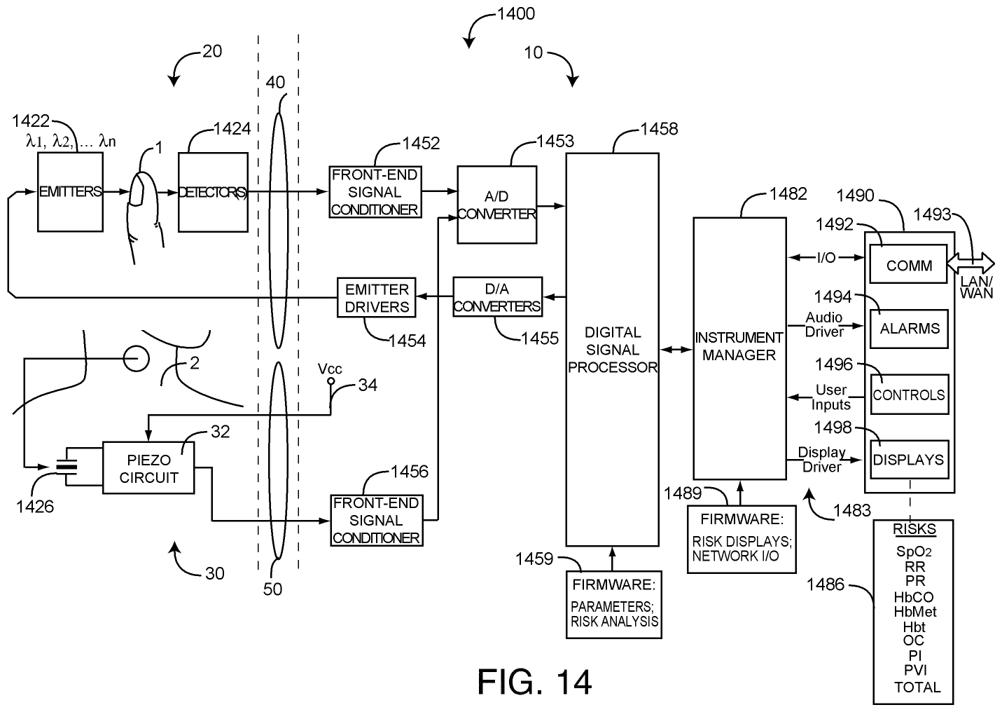

FIG. 14 illustrates a risk analysis system embodiment 1400 including an optical sensor 20 an acoustic sensor 30 and a monitor 10. The optical sensor 20 has LED emitters 1422 attached on one side of a fingertip site 1 and at least one detector 1424 attached on the opposite side of the fingertip site 1. In an embodiment, the emitters 1422 emit light having at least one red wavelength and at least one IR wavelength, so as to determine a ratio of oxyhemoglobin and deoxygemoglobin in the pulsatile blood perfused tissues of the fingertip. In other embodiments, the emitters 1422 generate light having more than two discrete wavelengths so as to resolve other hemoglobin components. Emitter drivers 1454 activate the emitters 1422 via drive lines in a sensor cable 40. The detector 1424 generates a current responsive to the intensity of the received light from the emitters 1422 after attenuation by the pulsatile blood perfused fingertip tissue 1. One or more detector lines in the sensor cable 40 transmit the detector current to the monitor 10 for signal conditioning 1452 and processing 1458, as described below. The acoustic sensor 30 has a piezo electric element 1426 attached to a neck site 2 and is in communications with a piezo circuit 32 and a power interface 34. The piezoelectric element 1426 senses vibrations and generates a voltage in response to the vibrations. The signal generated by the piezoelectric element 1426 is communicated to the piezo circuit 32, which transmits the signal via a sensor cable 50 to the monitor 10 for signal conditioning 1456 and processing 1458.

Also shown in FIG. 14, the monitor 10 drives and processes signals from an sensors 20, 30 and includes an optical front-end 1452, an acoustic front-end 1456, an analog-to-digital (A/D) converter 1453, a digital signal processor (DSP) 1458, emitter drivers 1454 and digital-to-analog (D/A) converters 1455. In general, the D/A converters 1455 and drivers 1454 convert digital control signals into analog drive signals capable of activating the emitters 1422 via a sensor cable 40. The optical front-end 1452 and A/D converter 1453 transform composite analog intensity signal(s) from light sensitive detector(s) received via the sensor cable 30 into digital data input to the DSP 1458. The acoustic front-end 1456 and A/D converter 1453 transform analog acoustic signals from a piezoelectric element into digital data input to the DSP 1458. The A/D converter 1453 is shown as having a two-channel analog input and a multiplexed digital output to the DSP 1458. In another embodiment, each front-end 1452, 1456 communicates with a dedicated single channel A/D converter generating two independent digital outputs to the DSP 1458. The DSP 1458 can comprise a wide variety of data and/or signal processors capable of executing programs 1459 for determining physiological parameters and risk.

Further shown in FIG. 14, an instrument manager 1482 communicates with the DSP 1458 to receive physiological parameter information derived by the DSP firmware 1459. One or more I/O devices 1490 have communications 1483 with the instrument manager 1482 including displays 1498, controls 1496, alarms 1494 and user I/O and instrument communication ports 1492. The alarms 1494 may be audible or visual indicators or both. The I/O 1492 may be, as examples, keypads, touch screens, pointing devices or voice recognition devices, to name a few. The I/O 1492 may also be any of various wired or wireless local area networks (LAN) or wide area networks (WAN) including the Masimo Patient SafetyNet (PSN) and the Internet. The displays 1498 may be indicators, numerics or graphics for displaying one or more of various physiological parameters 1459 or risk displays 1489 as described with respect to FIGS. 10-13, above, as examples. The instrument manager 1482 may also be capable of storing or displaying historical or trending data related to one or more of the measured values or combinations of the measured values.

A risk analysis system has been disclosed in detail in connection with various embodiments. These embodiments are disclosed by way of examples only and are not to be construed as limiting the scope of the claims that follow. One of ordinary skill in the art will appreciate many variations and modifications.

1. (canceled)

2. A patient monitoring system configured to generate a determined indication of risk output associated with increasing and immediate vulnerability to permanent physiological impairment or death of a monitored patient for a caregiver to provide treatment to the patient, the patient monitoring system comprising:
a physiological sensor comprising a plurality of LEDs and a detector, the plurality of LEDs configured to emit light at one or more wavelengths at a measurement site of a patient, the detector configured to generate physiological data associated with the one or more light wavelengths after attenuation by tissue at the measurement site of the patient; and
one or more hardware processors configured to:
for each of a plurality of different physiological parameters of the patient:
based at least in part on the physiological data, determine a plurality of different physiological parameter features, each feature associated with physiological phenomena that affect measurement of the physiological parameter, wherein the plurality of physiological parameter features is configured to allow accurate measurement of risk associated with the physiological parameter;
for each of the plurality of physiological parameter features:
determine a risk associated with the physiological parameter feature based on whether the physiological parameter feature is changing; and
determine a risk weight associated with the physiological parameter feature based on sensitivity of the physiological parameter feature risk to changes in the physiological parameter feature;
determine the risk associated with the physiological parameter based on each of the weighted physiological parameter feature risks; and
determine a risk weight associated with the physiological parameter based on sensitivity of total risk associated with the patient to changes in the physiological parameter;
determine the total risk associated with the patient based on each of the weighted physiological parameter risks, wherein the total risk indicates a level of urgency in clinical treatment and care of the patient; and
cause display of a visual indicator associated with the total risk in a display for visualization by a caregiver such that the caregiver is alerted for immediate attention to a condition of the patient.

3. The patient monitoring system of claim 2, wherein the plurality of physiological parameters includes one or more of at least: oxygen saturation, respiration rate, pulse rate, pulse rate variability index, and perfusion index.

4. The patient monitoring system of claim 2, wherein the plurality of physiological parameter features includes one or more of at least: a physiological parameter baseline, a physiological parameter instability, and an average slope associated with the physiological parameter.

5. The patient monitoring system of claim 4, wherein the one or more hardware processors are further configured to determine the physiological parameter baseline as riding near a top of a physiological parameter waveform over a period of time.

6. The patient monitoring system of claim 4, wherein the one or more hardware processors are further configured to determine the physiological parameter instability based on a plurality of deviations of a physiological parameter waveform from the physiological parameter baseline over a period of time.

7. The patient monitoring system of claim 4, wherein the one or more hardware processors are further configured to determine the average slope of the physiological parameter based on slopes of adjacent samples on a physiological parameter waveform over a period of time.

8. The patient monitoring system of claim 2, wherein the one or more hardware processors are further configured to determine a physiological parameter feature risk based on whether the physiological parameter feature is changing in a certain direction.

9. The patient monitoring system of claim 8, wherein the one or more hardware processors are further configured to determine a physiological parameter feature risk based on whether a physiological parameter feature value is either increasing or decreasing.

10. The patient monitoring system of claim 2, wherein the one or more hardware processors are further configured to determine one or more risk factors based on received patient data associated with the monitored patient.

11. The patient monitoring system of claim 10, wherein the one or more hardware processors are further configured to update one or more physiological parameter risks based on the one or more risk factors.

12. A patient monitoring system configured to generate a determined indication of risk output associated with a monitored patient in real-time to alert a caregiver to an increasing and immediate vulnerability of the monitored patient to permanent physiological impairment or death, the patient monitoring system comprising:
one or more hardware processors in communication with a physiological sensor having a plurality of LEDs configured to emit light of one or more wavelengths at a measurement site of a patient and at least one detector configured to generate at least one physiological signal associated with the one or more light wavelengths after attenuation by tissue at the measurement site of the patient; and
the one or more hardware processors configured to:
for each of a plurality of different physiological parameters of the patient:
based at least in part on the physiological signal, determine a plurality of different physiological parameter features, the plurality of physiological parameter features configured to allow accurate measurement of risk associated with the physiological parameter;
for each of the plurality of physiological parameter features:
determine a contribution to the physiological parameter risk based on a risk associated with the physiological parameter feature and a risk weight associated with the physiological parameter feature, wherein the physiological parameter feature risk is based on whether the physiological parameter feature is changing, and wherein the physiological parameter feature risk weight is based on sensitivity of the physiological parameter risk to changes in the physiological parameter feature; and
determine a contribution to a total risk associated with the patient based on a determined risk associated with the physiological parameter and a risk weight associated with the physiological parameter, wherein the physiological parameter risk is based on the contributions of each physiological parameter feature, and wherein the physiological parameter risk weight is based on sensitivity of the total risk to changes in the physiological parameter;
determine the total risk associated with the patient based on the contributions of each physiological parameter, wherein the total risk indicates a likelihood of serious illness or debilitation of the patient; and
cause display of a visual indicator associated with the total risk, the visual indicator configured to dynamically change responsive to a change in risk derived from one or more of the plurality of physiological parameters of the patient such that a caregiver is alerted for immediate attention to a change in condition of the patient.

13. The patient monitoring system of claim 12, wherein the plurality of physiological parameters includes one or more of at least: oxygen saturation, respiration rate, pulse rate, pulse rate variability index, and perfusion index.

14. The patient monitoring system of claim 12, wherein the plurality of physiological parameter features includes one or more of at least: a physiological parameter baseline, a physiological parameter instability, and an average slope associated with the physiological parameter.

15. The patient monitoring system of claim 14, wherein the one or more hardware processors are further configured to determine the physiological parameter baseline as riding near a top of a physiological parameter waveform over a period of time.

16. The patient monitoring system of claim 14, wherein the one or more hardware processors are further configured to determine the physiological parameter instability based on a plurality of deviations of a physiological parameter waveform from the physiological parameter baseline over a period of time.

17. The patient monitoring system of claim 14, wherein the one or more hardware processors are further configured to determine the average slope of the physiological parameter based on slopes of adjacent samples on a physiological parameter waveform over a period of time.

18. The patient monitoring system of claim 12, wherein the one or more hardware processors are further configured to determine a physiological parameter feature risk based on whether the physiological parameter feature is changing in a certain direction.

19. The patient monitoring system of claim 18, wherein the one or more hardware processors are further configured to determine a physiological parameter feature risk based on whether a physiological parameter feature value is either increasing or decreasing.

20. The patient monitoring system of claim 12, wherein the one or more hardware processors are further configured to determine one or more risk factors based on received patient data associated with the monitored patient.

21. The patient monitoring system of claim 20, wherein the one or more hardware processors are further configured to update one or more physiological parameter risks based on the one or more risk factors.
